(19) United States
(12) Patent Application Publication
(10) Pub. No.: US 2024/0368533 A1
(43) Pub. Date: Nov. 7, 2024

(54) METHOD OF PREPARING A BACTERIAL CULTURE

(71) Applicant: Chr. Hansen A/S, Hoersholm (DK)

(72) Inventors: Zuzana Mladenovska, Hoersholm (DK); Birgitte Yde, Hoersholm (DK); Hans Bisgaard-Frantzen, Hoersholm (DK)

(73) Assignee: Chr. Hansen A/S, Hoersholm (DK)

(21) Appl. No.: 18/687,536

(22) PCT Filed: Aug. 30, 2022
(86) PCT No.: PCT/EP2022/074050

§ 371 Date: Feb. 28, 2024

(30) Foreign Application Priority Data
Aug. 31, 2021 (EP) 21194077.0

(51) Int. Cl. C12N 1/20; C12N 1/04

(52) U.S. Cl. CPC C12N 1/20; C12N 1/04

(57) ABSTRACT

The present invention relates to the provision of methods of preparing a bacterial culture and bacterial cultures obtained thereby. The present invention also relates to the provision of methods for reducing viability loss and/or activity loss during freezing and/or drying of a bacterial culture and for increasing storage stability of a frozen and/or dried bacterial culture.

### TECHNICAL FIELD OF THE INVENTION

The present invention relates to the provision of methods of preparing a bacterial culture and bacterial cultures obtained thereby. The present invention also relates to the provision of methods for reducing viability loss and/or activity loss during freezing and/or drying of a bacterial culture and for increasing storage stability of a frozen and/or dried bacterial culture.

### BACKGROUND OF THE INVENTION

Before inoculation of a bacterial culture into products, such as food products, bacteria are cultured in order to provide a suspension containing large amounts of bacteria. Cultures of fermentative bacteria are typically produced by fermentation processes with acidic pH, typically in the range of pH 3.0 to pH 6.5. After termination of the fermentation process, the suspension is usually concentrated. This concentration step is often followed by freezing the bacterial concentrate as a frozen product in liquid nitrogen and optionally further freeze-drying the frozen bacterial concentrate, to preserve and/or store the bacteria. Alternatively the concentration step is often followed by drying the bacterial concentrate before storage.

However, downstream processing steps, particularly freezing and/or drying, can hinder the industrial production of storable viable bacteria due to the cell damage and loss of viable cells during these steps. Freeze-drying can take multiple days while other process steps typically take a few hours.

Thus, there is an unmet need for improving the robustness of bacterial cultures such that they can better withstand the downstream processing steps after termination of fermentation.

Hence, it would be advantageous to provide improved methods for preparing a bacterial culture and bacterial cultures obtained thereby. In particular, it would be advantageous to provide methods for reducing viability loss and/or activity loss during freezing and/or drying of a bacterial culture and for increasing storage stability of a frozen and/or dried bacterial culture.

### SUMMARY OF THE INVENTION

The present invention relates to methods of preparing a bacterial culture in which the pH is adjusted to a pH in the range of pH 6.0 to pH 8.0 at a stage after termination of fermentation. In particular, the present invention discloses bacterial cultures prepared by such methods. Accordingly, the present invention provides methods of preparing a bacterial culture, methods for reducing viability loss and/or activity loss during freezing and/or drying of a bacterial culture, methods for increasing storage stability of a frozen and/or dried bacterial culture, and bacterial cultures produced by such methods.

The present inventors have discovered that raising the pH of a bacterial culture after termination of fermentation, or after a downstream processing step of concentrating the bacterial culture or adding a protective compound to the concentrated bacterial culture, results in the bacterial cells exhibiting better activity and/or viability during subsequent downstream processing steps than bacterial cells processed from bacterial cultures with pH less than 6.0. As shown herein, the methods of the present invention enable an improvement of general cell robustness and reduction of losses in viability and/or activity during freezing and freeze-drying. For example, the bacterial cells show increased maintenance of cell integrity and active metabolism. Further, freeze-dried products with the post-fermentation adjusted pH have improved stability during shelf storage at elevated temperature or water activity greater than 0.1.

Hence, for the first time, the inventors have surprisingly shown that raising the pH of a bacterial culture at a stage after termination of fermentation is an efficient means of improving the overall robustness of cells.

This new invention is inexpensive to implement and is applicable to a wide variety of cultures of fermentative bacteria. It can therefore be applied to improve the quality and performance of bacterial cultures, including in frozen, freeze-dried and dried formats.

Thus, a first aspect of the present invention relates to a method of preparing a bacterial culture, the method comprising the steps of:
(a) culturing fermentative bacteria in a fermentation medium with a pH lower than pH 6.0 at the point of termination of fermentation and obtaining a bacterial culture,
(b) optionally concentrating the bacterial culture by a one-step concentration method to obtain a concentrated bacterial culture, and
(c) further optionally adding a protective compound to the concentrated bacterial culture of step (b);
   wherein the pH is adjusted to a pH in the range of pH 6.0 to pH 8.0 following step (a), (b) or (c).

A second aspect of the present invention relates to a bacterial culture obtained by a method according to the first aspect of the invention.

A third aspect of the present invention relates to a method for reducing viability loss and/or activity loss during freezing and/or drying of a bacterial culture, wherein the method comprises the steps of:
(a) culturing fermentative bacteria in a fermentation medium with a pH lower than pH 6.0 at the point of termination of fermentation and obtaining a bacterial culture;
(b) concentrating the bacterial culture by a one-step concentration method to obtain a concentrated bacterial culture; and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

A fourth aspect of the present invention relates to a method for increasing storage stability of a frozen and/or dried bacterial culture, wherein the method comprises the steps of:
(a) culturing fermentative bacteria in a fermentation medium with a pH lower than pH 6.0 at the point of termination of fermentation and obtaining a bacterial culture;
(b) concentrating the bacterial culture by a one-step concentration method to obtain a concentrated bacterial culture; and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

### BRIEF DESCRIPTION OF THE FIGURES

FIG. 1 shows the reduction of viable cells of freeze-dried granulates of Lactobacillus acidophilus bacterial cultures over a storage period of 16 weeks in aluminium pouches at 30° C., measured by loss of colony forming units (CFU) on a log scale. Comparison was made between freeze-dried granulates from bacterial cultures having post-concentration pH increase to pH 6.5 and freeze-dried granulates without post-concentration pH increase (maintenance at pH<5.0).

The present invention will in the following be described in more detail.

### DETAILED DESCRIPTION OF THE INVENTION

A first aspect of the present invention provides a method of preparing a bacterial culture, the method comprising the steps of:
(a) culturing fermentative bacteria in a fermentation medium with a pH lower than pH 6.0 at the point of termination of fermentation and obtaining a bacterial culture,
(b) optionally concentrating the bacterial culture by a one-step concentration method to obtain a concentrated bacterial culture, and
(c) further optionally adding a protective compound to the concentrated bacterial culture of step (b);
   wherein the pH is adjusted to a pH in the range of pH 6.0 to pH 8.0 following step (a), (b) or (c).

In the present context, the term “bacterial culture” refers to a population of bacteria.

“Fermentation” in the method of the present invention is to be understood as the conversion of carbohydrates into alcohols or acids through the action of a microorganism. Preferably, fermentation in the methods of the invention comprises conversion of lactose to lactic acid.

“Fermentative bacteria” are to be understood as bacteria that are capable of fermentation.

A “fermentation medium” is to be understood as a liquid medium for the growth of fermentative bacteria. Such a medium would typically contain the necessary nutrients for growth of the fermentative bacteria. Preferably the fermentation medium contains appropriate nutrients for the particular fermentative bacteria to be cultured. A wide variety of fermentation media are well known in the art, including the suitability of fermentation media for a given bacterial species to be cultured.

Methods for culturing fermentative bacteria in a fermentation medium are also well known in the art, as are culture vessels for use in such methods. For example, the culture vessel could be a sealed bottle or a conical flask, optionally to be shaken with a rotary shaker, or an industrial fermenter or bioreactor.

“Termination of fermentation” is understood to mean the point at which the fermentation process is ended. For example, this could be because the bacterial culture enters a holding period (e.g. when bacteria reached stationary growth phase, or when carbohydrates present in the fermentation media have been metabolized) or is processed as a non-fermentative step, such as concentration, freezing, drying etc. Typically, it would be appropriate to terminate the fermentation after it has reached an optical density measured at 600 nm wavelength (OD₆₀₀) of at least 1, preferably an OD₆₀₀ of 4-60. Suitable methods and apparatuses for measuring optical density or turbidity are well known in the art, such as a spectrophotometer.

In accordance with the present invention, fermentative bacteria are cultured and the final pH of the fermentation broth at the point of termination of fermentation is at a pH lower than pH 6.0, for example at pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, pH 4.4, pH 4.3, pH 4.2, pH 4.1, pH 4.0, pH 3.9, pH 3.8, pH 3.7, pH 3.6, pH 3.5, pH 3.4, pH 3.3, pH 3.2, pH 3.1. pH 3.0 or lower. Culturing the fermentative bacteria with a pH lower than pH 6.0 at the point of termination of fermentation can be understood to include (i) use of a means of pH control to achieve an essentially constant pH level in the culture during the fermentation process, (ii) starting at a pH greater or equal to pH 6.0 and not restricting the natural acidification of the medium by the bacteria during the fermentation process, or (ii) starting at a pH lower than pH 6.0 and not restricting the natural acidification of the medium by the bacteria during the fermentation process.

For example, from the beginning to the termination of said fermentation in step (a), the pH decreases to no more than pH 6.0, such as no more than pH 5.5, no more than pH 5.0, no more than pH 4.5, no more than pH 4.0, no more than pH 3.5, no more than pH 3.0 and/or decreases by at least 0.1 pH units, preferably at least 0.2, 0.5, 1.0, 1.5, 2.0, 2.5, 3.0, 3.5 or 4.0 pH units.

The invention involves adjustment of the pH of the bacterial culture to a less acidic, neutral or mildly alkaline pH at a stage after termination of fermentation chosen from step (a), step (b) or step (c) above. In accordance with the invention, the pH is adjusted to within the range of pH 6.0 to pH 8.0. For example, the pH may be adjusted to pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9 or pH 8.0.

pH adjustment is typically carried out by the addition of alkali or base to the bacterial culture when it is in the form of a suspension of bacterial cells. Any suitable alkali or base can be used. Preferably, the alkali or base is one that is not toxic to the bacteria nor to eukaryotes, such as mammals, so that it can be used safely without compromising the bacterial culture or any subsequent use thereof.

Hence, most preferably, the pH adjustment following step (a), (b) or (c) is by adding a base selected from the group consisting of ammonium hydroxide, sodium hydroxide, potassium hydroxide and sodium carbonate.

In one embodiment, the adjustment of pH takes place after termination of fermentation (i.e. after step (a)) but before any additional manipulation steps. In one embodiment, a holding period immediately follows the termination of fermentation and the adjustment of pH takes place at the end of the holding period. “Holding period” is to be understood as a period between termination of fermentation and start of the downstream process.

In some embodiments, the method further comprises step (b): concentrating the bacterial culture by a one-step concentration method to obtain a concentrated bacterial culture. In some of these embodiments, the adjustment of pH is carried out on said concentrated bacterial culture, as an alternative to doing so after step (a).

A “concentrated bacterial culture” is to be understood as a bacterial culture that, as a result of a concentration method, has a higher optical density of the cells suspended in it compared to the bacterial culture before carrying out the concentration method, without increasing the total number of cells. In other words, the number of cells remains essentially constant while the volume of the bacterial culture suspension is reduced. A concentrated bacterial culture typically has an OD₆₀₀ of 50-800. A concentrated bacterial culture typically has a dry matter content of 8% to 28%. A concentrated bacterial culture typically contains 1×10⁹ to 1×10¹³ total cells per gram of concentrated bacterial culture. A “one-step concentration method” is to be understood as a concentration step that produces a concentrated bacterial culture in one step, and wherein the cells are maintained in suspension during the entire concentration step.

For the avoidance of doubt, a one-step concentration method does not include the two-step method of (i) formation of a pellet of bacterial cells and (ii) resuspension of said cell pellet. The use of a one-step concentration method has the advantage of, inter alia, preventing changes to the cells that would result from bringing them out of suspension in the culture medium, and may enable the procedure to be performed on an industrial scale.

Preferably, step (b) is carried out by a technique selected from the group consisting of centrifugal separation, vacuum evaporation and filtration.

In some embodiments, the method further comprises step (c): adding a protective compound to the concentrated bacterial culture of step (b). A “protective compound” is to be understood as any compound that provides a protective effect to the bacterial culture during further steps of industrial processing and storage. In some of these embodiments, the adjustment of pH is carried out on said concentrated bacterial culture containing said protective compound, as an alternative to doing so after any of steps (a) and (b).

The bacterial culture of the present invention may be provided in several forms. It may be a frozen form, dried form, freeze dried form, or liquid form. It may be a powder, pellets or tablets. Thus, in one embodiment the composition is in frozen, dried, freeze-dried or liquid form.

In some embodiments, the method comprises steps (a) and (b), or steps (a), (b) and (c), and further comprises step (d): freezing the concentrated bacterial culture to obtain a frozen bacterial culture. Hence, in such embodiments, the method may comprise steps (a), (b) and (d); or steps (a), (b), (c) and (d).

Freezing the concentrated bacterial culture can be done by any suitable method known in the art. Typically, concentrated bacterial cultures are frozen by pelletizing cell concentrate in liquid nitrogen. Frozen concentrated bacterial cultures are typically stored at about −55° C. Preferably, said concentrated bacterial culture comprises a cryoprotectant as the protective compound added in step (c).

In some embodiments, the method further comprises step (e): removing water from said concentrated bacterial culture or frozen bacterial culture to obtain a dried bacterial culture. Hence, in such embodiments, the method may comprise steps (a), (b) and (e); steps (a), (b), (c) and (e); steps (a), (b), (d) and (e); or steps (a), (b), (c), (d) and (e).

Removing water can be done by any suitable method known in the art. Preferably, step (e) is carried out by a technique selected from the group consisting of spray drying, spray freezing, vacuum drying, air drying, freeze drying, tray drying and vacuum tray drying.

The dried bacterial culture may be in the form of a granulate and/or a powder. For example, the dried bacterial culture may be in the form of a spray-dried powder, a freeze-dried granulate or a freeze-dried powder.

In some embodiments, the method further comprises step (f): packing said frozen bacterial culture obtained in step (d) or said dried bacterial culture obtained in step (e). Hence, in such embodiments, the method may comprise steps (a), (b), (d) and (f); steps (a), (b), (c), (d) and (f); steps (a), (b), (e) and (f); steps (a), (b), (c), (e) and (f); steps (a), (b), (d), (e) and (f); or steps (a), (b), (c), (d), (e) and (f).

The method of the invention is widely applicable. Thus, in one embodiment the bacterial culture comprises or consists of fermentative bacteria:
from the phylum Firmicutes, such as:
   a lactic acid bacterium (LAB), preferably of a genus selected from the group consisting of Streptococcus (such as Streptococcus thermophilus), Lactococcus (such as Lactococcus lactis), Oenococcus (such as Oenococcus oeni), Leuconostoc (such as species Leuconostoc mesenteroides, Leuconostoc pseudomesenteroides), Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus; Eubacterium (such as Eubacterium limosum, Eubacterium aggregans, Eubacterium barkeri, Eubacterium lentum), Roseburia (Roseburia intestinalis, Roseburia hominis, Roseburia inulinivorans, Roseburia faecis and Roseburia cecicola), Faecalibacterium (such as species Faecalibacterium prausnitzii), Anaerostipes (such as Anaerostipes cacccae), Anaerobutyricum (such as species Anaerobutyricum hallii, Anaerobutyricum soehngenii)
   from the phylum Actinobacteria, such as genus Bifidobacterium (such as species Bifidobacterium animalis, Bifidobacterium longum, Bifidobacterium adolescentis, Bifidobacterium breve), genus Propionibacterium (such as species Propionibacterium freudenreichii), Cutibacterium (such as Cutibacteriun acnes)
   from the phylum Bacteroidetes, such as genera Bacteroides (such as species Bacteroides fragilis, Bacteroides xylanisolvens), Prevotella (such as species Prevotella copri) or Alistipes, and/or
   from the phylum Verrucomicrobia, such as an Akkermansia (such as species Akkermansia muciniphila).

In particular, the bacteria can be one or more of: Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

The method is applicable to vegetative cells of non-spore-forming bacteria from the domain Bacteria. The invention relates to a broad spectrum of non-sporulating bacteria used in food- and feed-producing industries, agriculture, medicine, for production of biofuels and biobased chemicals.

Non-spore-forming bacteria can be identified within the phyla Firmicutes, Actinobacteria and Bacteroidetes. The invention is particularly applicable to homo- and heterofermentative lactic acid bacteria in the Firmicutes phylum, and to bifidobacteria and propionibacteria in the Actinobacteria phylum. The invention is also applicable to obligate anaerobes of the class Clostridia in the Firmicutes phylum, such as fermentative, butyrate-producing bacteria of the genera Roseburia (e.g. Roseburia hominis and Roseburia inulinivorans), Anaerobutyricum hallii, Anaerobutyricum soehngenii, Eubacterium (e.g. Eubacterium limosum), Anaerostipes (e.g. Anaerostipes caccae), and Faecalibacterium (e.g. F. prausnitzii) which represent the core microbiota of human intestinal tract and are candidates for next generation of probiotics.

In the present context, the term “lactic acid bacteria (LAB)” refers to a group of Gram positive, catalase negative, non-motile, microaerophilic or anaerobic bacteria that ferment sugar with the production of acids including lactic acid as the predominantly produced acid, acetic acid, formic acid and propionic acid. The industrially most useful lactic acid bacteria include, but are not limited to, Lactococcus species (spp.), Streptococcus spp., Lactobacillus spp. (including all those that were classed as Lactobacillus until 2020), Leuconostoc spp., Pediococcus spp., Brevibacterium spp, Enterococcus spp. and Propionibacterium spp. Additionally, lactic acid producing bacteria belonging to the group of the strict anaerobic bacteria, Bifidobacteria, i.e. Bifidobacterium spp. which are frequently used as food starter cultures alone or in combination with lactic acid bacteria, are generally included in the group of lactic acid bacteria. Even certain bacteria of the genus Staphylococcus (e.g. S. carnosus, S. equorum, S. sciuri, S. vitulinus and S. xylosus) have been referred to as LAB (Mogensen et al (2002)).

It will be appreciated (and is mentioned above) that the Lactobacillus genus taxonomy was updated in 2020. The new taxonomy is disclosed in Zheng et al. 2020 and will be cohered to herein in the absence of an indication to the contrary. For the purpose of the present invention, Table 1 presents a list of new and old names of some Lactobacillus species relevant to the present invention.

TABLE 1
New and old names of some Lactobacillus
species relevant to the present invention

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei | Lacticaseibacillus paracasei |
| subsp. paracasei | subsp. Paracasei |
| Lactobacillus plantarum | Lactiplantibacillus plantarum |
| subsp. plantarum | subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

The lactic acid bacteria are preferably of a genus selected from the group consisting of Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lacti-plantibacillus. In particular, they can be Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, and/or Lactiplantibacillus pentosus. Others include Lactococcus lactis subsp. lactis, Lactococcus lactis subsp. cremoris, Leuconostoc lactis, Leuconostoc mesenteroides subsp. cremoris, Pediococcus pentosaceus, Lactococcus lactis subsp. lactis biovar. diacetylactis, Streptococcus thermophilus, Enterococcus, such as Enterococcus faecium, Bifidobacterium animalis subsp. lactis, Bifidobacterium animalis subsp. animalis, Bifidobacterium longum, Bifidobacterium adolescentis, Bifidobacterium breve, Lactobacillus helveticus, Lactobacillus fermentum, Lactobacillus salivarius, Lactobacillus delbrueckii subsp. bulgaricus and Lactobacillus acidophilus.

The composition may comprise one or more strains of lactic acid bacteria which may be selected from the group comprising: BB-12® (Bifidobacterium animalis subsp lactis BB-12®, DSM 15954), ATCC 29682, ATCC 27536, DSM 13692, DSM 10140, LA-5 (Lactobacillus acidophilus LA-50®, DSM 13241), LGG® (Lactobacillus rhamnosus LGGR, ATCC 53103), GR-1® (Lactobacillus rhamnosus GR-1®, ATCC 55826), RC-14® (Lactobacillus reuteri RC-14®, ATCC 55845), L. casei 431® (Lactobacillus paracasei subsp. paracasei L. casei 431®, ATCC 55544), F19® (Lactobacillus paracasei F19®, LMG P-17806_, TH-4® (Streptococcus thermophilus TH-4®, DSM 15957), PCC® (Lactobacillus fermentum PCC®, NM02/31074), LP-33® (Lactobacillus paracasei subsp. paracasei LP-33®), Lactococcus lactis DSM 21404, Ligilactobacillus animalis DSM 33570, Bifidobacterium animalis subsp. lactis DSM 33868, and CCTCC M204012.

The LAB culture may be a “mixed lactic acid bacteria (LAB) culture” or a “pure lactic acid bacteria (LAB) culture”. The term “mixed lactic acid bacteria (LAB) culture”, or “LAB” culture, denotes a mixed culture that comprises two or more different LAB species. The term a “pure lactic acid bacteria (LAB) culture” denotes a pure culture that comprises only a single LAB species. Accordingly, in a preferred embodiment the LAB culture is a LAB culture selected from the group consisting of these cultures.

Preferably, the LAB cell is a probiotic cell.

It will be appreciated that any suitable fermentation medium can be used for culturing the fermentative bacteria in step (a). For example, modified Difco™ M17 Broth (Becton, Dickinson and Company, France) contains peptones and meat derivatives as sources of carbon, nitrogen, vitamins and minerals; yeast extract supplies B-complex vitamins which stimulate bacterial growth; disodium-β-glycerophosphate buffers the medium while acid is produced from the fermentation of lactose; ascorbic acid stimulates growth of lactic Streptococci. Difco™ Lactobacilli MRS Broth powder contains peptone and dextrose, which supply nitrogen, carbon and other elements necessary for growth; Polysorbate 80, acetate, magnesium and manganese provide growth factors for culturing a variety of Lactobacilli.

It is highly preferred that the fermentation medium comprises at least one carbohydrate. Preferably, said carbohydrate is one or more of: a monosaccharide such as glucose, fructose, galactose or mannose; a disaccharide such as sucrose, trehalose, maltose or lactose; a sugar alcohol such as inositol; a trisaccharide such as maltotriose or raffinose; an oligosaccharide such as a fructooligosaccharide or such as a maltodextrin with DE 3-20; a glucose syrup with DE 21-39; and a polysaccharide such as starch or inulin.

Fructo-oligosaccharides (FOS), also known as oligofructose or oligofructan, are mixtures of oligosaccharide fructans, and are typically prepared by the transfructosylation action of a β-fructosidase of Aspergillus niger or Aspergillus on sucrose.

Inulin is a heterogeneous collection of fructose polymers and can be employed in the invention in various forms, for example granules and powders, which are commercially available.

Maltodextrin is a polysaccharide that consists of D-glucose units connected in chains of variable length. The glucose units are primarily linked with a (1-+4) glycosidic bonds. Maltodextrin is typically composed of a mixture of chains that vary from three to 17 glucose units long. Maltodextrins are classified by DE (dextrose equivalent) and have a DE between 3 and 20, preferably between 10 and 20. The higher the DE value, the shorter the glucose chains, the higher the sweetness, the higher the solubility, and the lower heat resistance.

For growth of LAB, the fermentation medium preferably comprises a simple carbohydrate such as a monosaccharide.

Preferably the total concentration of the carbohydrate in the fermentation medium in step (a) is 1-15% w/w, such as 2-14% w/w, e.g. 3-13% w/w, such as 4-12% w/w, e.g. 5-11% w/w, such as 6-10% w/w, e.g. 7-9% w/w, such as 8-10% w/w, more preferably 1-10% w/w.

Preferably the fermentation medium in step (a) comprises at least one nitrogen source, such as peptones, yeast extract, one or more amino acids or one or more ammonia salts.

Preferably the fermentation medium in step (a) comprises one or more yield enhancing agents selected from the group consisting of a purine base, a pyrimidine base, a nucleoside, a nucleotide and derivatives thereof.

Herein, the term “purine base” is intended to cover a cyclic nitrogen-containing base having the core structure of purine. Thus, in the present context, the term “purine base” is intended to mean an optionally substituted purine. Specific examples of purine bases include adenine, guanine, xanthine and hypoxanthine. Analogously, the term “pyrimidine base” is intended to cover a cyclic nitrogen-containing base having the core structure of pyrimidine. Thus, in the present context, the term “pyrimidine base” is intended to mean an optionally substituted pyrimidine. Specific examples of pyrimidine bases include cytosine, thymine and uracil.

In the present context the term “nucleotide” means a 2-deoxyribose (DNA) monomer or a ribose (RNA) monomer which is bonded through its number one carbon atom to a purine base or a pyrimidine base. Further, the DNA or RNA monomer is bonded through its number five-carbon atom to a phosphate group. When used herein, the term “nucleoside” is intended to mean a 2-deoxyribose (DNA) monomer or a ribose (RNA) monomer which is bonded through its number one carbon atom to a purine base or a pyrimidine base. In the present context, the term “derivative”, when used in connection with the terms “nucleotide” or “nucleoside” is intended to mean that the nucleotide or the nucleoside in question has been modified in its sugar (i.e. 2-deoxyribose or ribose) unit, or that the nucleotide or the nucleoside in question has been modified in its cyclic nitrogen-containing base, or that the nucleotide or nucleoside in question has been modified in both its sugar unit and in its cyclic nitrogen-containing base.

Preferably, the fermentation medium in step (a) comprises one or more vitamins. Fermentation medium can also comprise a source of fatty acid, such as oleic acid in Polysorbate 80.

As discussed above, optional step (c) provides further adding a protective compound to the concentrated bacterial culture of step (b). Said protective compound may be selected from the group consisting of cryoprotectants, lyoprotectants, antioxidants, nutrients, fillers, flavorants and mixtures thereof. The bacterial culture obtained by the present invention may therefore comprise one or more of cryoprotectants, lyoprotectants, antioxidants and/or nutrients, more preferably cryoprotectants, lyoprotectants and/or antioxidants and most preferably cryoprotectants or lyoprotectants, or both. A concentrated bacterial culture to which one or more protective compound(s) has been added is sometimes referred to herein as a formulated bacterial culture.

Use of protectants such as cryoprotectants and lyoprotectant are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate).

In one embodiment, the bacterial culture according to the present invention comprises one or more cryoprotectants or lyoprotectants selected from the group consisting of: inosine-5′-monophosphate (IMP), adenosine-5′-monophosphate (AMP), guanosine-5′-uranosine-5′-monophosphate monophosphate (GMP), (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants for use in the invention include ascorbic acid, citric acid and salts thereof, gallates, cysteine, vitamin E, β-carotene and other carotenoids. Suitable nutrients for use in the invention include sugars, amino acids, fatty acids, minerals, trace elements, vitamins (such as vitamin B-family, vitamin C). The bacterial culture may optionally comprise further substances including fillers (such as lactose, maltodextrin or milk powder) and/or flavorants.

In a further aspect, the bacterial culture contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc., should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH3), NH₄OH, NH4+, and the like.

The amount of protective compound or mixture of protective compounds added to the bacterial culture is typically calculated as the dry weight of the protective compound(s) as a percentage of the dry weight of the formulated bacterial culture, i.e. the total dry weight of the biomass of the concentrated bacterial culture and the protective compound(s). It should be noted that the protective compound(s) are typically used in the form of a solution that is added to the concentrated bacterial culture (in the form of a suspension of bacterial cells). Thus, for example, adding the protective compound(s) at 50% w/w means adding the same amount of dry weight of protective compound(s) as the dry weight of the concentrated bacterial culture, i.e. a 1:1 ratio. Preferably, the concentration of the protective compound in the formulated bacterial culture in step (c) is 5-90% w/w assessed as dry weights, more preferably 25-75% w/w assessed as dry weights, more preferably 50-75% w/w assessed as dry weights. As for all embodiments of the invention described herein, it should be recognized that the ranges may be increments of the described ranges.

As the present invention is widely applicable, it will be appreciated that the fermentation in step (a) can vary in its parameters. In some embodiments, said fermentation in step (a) lasts from about 4 hours to about 7 days.

Preferably, the temperature of the fermentation in step (a) will be particularly suitable for growth of the fermentative bacteria. For example, the temperature may be the known optimum temperature for growth of the fermentative bacteria. In some embodiments, said fermentation in step (a) is at a temperature of from about 25° C. to about 50° C.; for example, 25° C., 26° C., 27° C., 28° C., 29° C., 30° C., 31° C., 32° C., 33° C., 34° C., 35° C., 36° C., 36.5° C., 37° C., 37.5° C., 38° C., 39° C., 40° C., 41° C., 42° C., 43° C., 44° C., 45° C., 46° C., 47° C., 48° C., 49° C. or 50° C.

In some embodiments, the termination of fermentation in step (a) is immediately followed by a holding period of from about 1 minute to about 10 hours. Preferably, during said holding period the bacterial culture is held at 4° C. to 45° C., more preferably 5° C. to 37° C., 6° C. to 25° C., or even more preferably 7° C. to 15° C.

In some embodiments, the dried bacterial culture obtained in step (e) has a water activity (a_{w}) in the range from 0.01-0.8, preferably in the range from 0.05-0.6, more preferably in the range from 0.05-0.4.

In the present context, the term “water activity” refers to the partial vapour pressure of water in a substance divided by the standard state partial vapour pressure of water. The water activity is denoted a_{w}. Specifically, a_{w} of a product (e.g. dried bacterial culture) is the ratio between the vapor pressure of the dried bacterial culture itself, when in a completely undisturbed balance with the surrounding air media, and the vapor pressure of distilled water under identical conditions.

For example, as in the Examples provided herein, water activity (a_{w}) of the freeze-dried material may be measured at room temperature using an Rotronic HYGROMER®) AwVC (Rotronic Instrument Corp., Huntington, NY, USA). The range of measurement that is achievable by use of this equipment corresponds to a water activity in the range of 0.03 to 1, and thus has an equipment Limit of Detection (LOD) of a water activity of 0.03.

In some embodiments, the frozen bacterial culture comprises in the range from 10⁴-10¹² colony forming units (CFU) per gram of frozen bacterial culture. In some embodiments, the dried bacterial culture comprises in the range from 10⁴-10¹³CFU/g dried bacterial culture. For example, the frozen and/or dried bacterial cultures may comprise in the range of from 108 to 10¹²CFU/g, or 10⁹ to 1011 CFU/g, or 10⁹ to 1010 CFU/g.

In the present context, and as shown in the present Examples, the robustness and survival of the bacterial cells in a bacterial culture can be determined by at least the following methods.

(1) Counts of total cells and active cells can be performed by flow cytometry. As demonstrated in the present Examples, sample preparation and flow cytometric assay can be carried out according to disclosure of EP 1 891 436 B1 (Worm et al) in paragraphs to [0061]. Active cells are characterized by their capability to maintain cell wall integrity, activation of cell metabolism, and cell membrane potential. Such results are a good guide to the degree of preservation of the cells.

(2) Enumeration of viable cells by colony counts. In the present context, the terms “viable” and “viability” refer to the ability of bacteria to reproduce. Thus, viability can be assessed by viability assays such as the determination of colony forming units. This can be done with any bacterial culture, but for the purpose of the present Examples this was determined for frozen and dried products after pelleting in liquid nitrogen and freeze-drying, respectively. Standard pour-plating method is used. The materials are suspended in sterile peptone saline diluent and homogenized by stomaching. After 30 minutes of revitalization, stomaching is repeated and the cell suspension is serially diluted in peptone saline diluent. The dilutions are plated in duplicates on appropriate agars. The agar plates are incubated anaerobically for three days at optimum growth temperatures of bacteria tested. Plates with 30-300 colonies are chosen for counting of colony forming units (CFU). The result is reported as average CFU/g sample, calculated from the duplicates.

(3) Determining metabolic activity in a food product. Viability and metabolic activity are not synonymous concepts. Commercial frozen or freeze-dried cultures may retain their viability, although they may have lost a significant portion of their metabolic activity e.g. cultures may lose their acid-producing (acidification) activity when kept stored even for shorter periods of time. Thus viability and metabolic activity have to be evaluated by different assays. Viability is typically assessed by viability assays such as the determination of colony forming units, whereas metabolic activity is assessed by quantifying the relevant metabolic activity of the bacterial culture. For example, metabolic activity may be measured as the acidification rate of milk inoculated with the bacterial culture. The term “metabolic activity” refers to the oxygen removal activity of the cultures, its acid-producing activity, i.e. the production of e.g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In the present context, and as shown in the present Examples, the storage stability of a sample (e.g. liquid or dry) is assessed by counting the colony-forming units (CFU) per gram, using the viability assay above.

Thus, in some embodiments, the dried bacterial culture comprises a content of viable fermentative bacteria in the range from 10⁴-10¹³ CFU/g dried bacterial culture after storage for 16 weeks at 30° C. and a_{w}=0.3.

In some embodiments, the loss of viability of the dried bacterial culture as measured by CFU/g is less than 4 log units after storage for 16 weeks at 30° C. and a_{w}=0.3, preferably less than 3 log units, more preferably less than 2 log units, even more preferably less than 1 log units or less than 0.5 log units. Most preferred is where there is no loss of viability, i.e. 0 log units.

Viability of dried bacterial cultures (such as freeze-dried bacterial cultures) can be measured over time to determine storage stability, such as using the following method that was demonstrated in the stability trials of the present Examples. Bacterial cultures were sampled immediately after initiation and at selected time points during the storage stability studies. Stability of bacterial cultures were assessed from the difference between CFU/g measured at the time 0 of the storage stability trials and at the specific sampling points of the stability test period. Loss of viability was quantified as CFU log loss.

A second aspect of the invention provides a bacterial culture obtained by a method according to the first aspect of the invention.

A third aspect of the present invention provides a method for reducing viability loss and/or activity loss during freezing and/or drying of a bacterial culture, wherein the method comprises the steps of:
(a) culturing fermentative bacteria in a fermentation medium with a pH lower than pH 6.0 at the point of termination of fermentation and obtaining a bacterial culture,
(b) concentrating the bacterial culture by a one-step concentration method to obtain a concentrated bacterial culture, and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

The features of the third aspect of the invention are in accordance with the corresponding features of the first aspect of the invention.

In the present context, the term “viability loss” refers to a reduction in the viability of a bacterial culture. For example, this may be expressed as a reduced number of colony forming units per gram of the bacterial culture. Thus, in the present context “reducing viability loss” means to reduce the proportion of bacterial cells that become unviable as a result of one or more process steps, particularly during the freezing and/or drying of the bacterial culture.

In the present context, the term “activity loss” refers to a reduction in the number of cells of a bacterial culture that are active. Thus, in the present context “reducing activity loss” means to reduce the proportion of bacterial cells that are no longer active as a result of one or more process steps, particularly during the freezing and/or drying of the bacterial culture.

A fourth aspect of the present invention provides a method for increasing storage stability of a frozen and/or dried bacterial culture, wherein the method comprises the steps of:
(a) culturing fermentative bacteria in a fermentation medium with a pH lower than pH 6.0 at the point of termination of fermentation and obtaining a bacterial culture,
(b) concentrating the bacterial culture by a one-step concentration method to obtain a concentrated bacterial culture; and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

The features of the fourth aspect of the invention are in accordance with the corresponding features of the first aspect of the invention.

In the present context, the term “storage stability” refers to the ability of a bacterial culture to maintain viability when stored over an extended duration of time. Typically, storage stability studies are conducted at ambient temperature (25° C.) for a period up to 2 years. Accelerated storage stability studies can be conducted at elevated water activity (a_{w}), elevated temperature, or combinations of elevated a_{w} and temperature. Decay of bacterial cells is expected to be faster under accelerated storage conditions.

Examples of elevated temperatures are 30° C. or 37° C., and elevated a_{w} denotes a_{w} higher than 0.1. For example, storage stability may be measured at a temperature of 30° C. and a_{w}≤0.15 for a period of 4 weeks or 8 weeks, or at a temperature of 30° C. and a_{w}=0.30 for a period up to 24 weeks (such as 8, 12, 16 20 or 24 weeks).

Storage stability can be determined by analysing how the count of viable bacterial cells develops over time. Viability of the bacterial culture is measured by determining the CFU/g as described herein. Thus, a measure of the storage stability of the bacterial culture may be determined by evaluating CFU/g of the dried bacterial culture at time point 0 (just after drying) and after e.g. 4 weeks of storage at accelerated storage conditions.

In brief, the storage stability of the dried bacterial culture may be investigated as follows, including as demonstrated in the present Examples:
(1) Freeze-dried granulates with a_{w}<0.15 are sealed in aluminium pouches and incubated at appropriate temperature for the desired period and CFU/g is determined for the samples.
(2) Freeze-dried granulates with a_{w}<0.15 are aliquoted in aluminium pouches and not sealed, then incubated open in atmospheric air at appropriate temperature and relative humidity for the desired period and CFU/g is determined for the samples.
(3) Freeze-dried granulates are grinded to powders and blended in microcrystalline cellulose equilibrated to a specific water activity (a_{w}). The samples are placed in aluminium bags and the bags are sealed. The bags are stored at appropriate temperature for the desired period and CFU/g is determined for the samples.

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. Embodiments and features of the present invention are also outlined in the following items.

The invention will now be described in further details in the following non-limiting examples.

### EXAMPLES

### Example 1: Increased activity of Lactococcus lactis cultures with post-fermentation pH increase

This Example involves the culture and downstream processing of a Lactococcus lactis strain deposited under accession number DSM 21404 at DSMZ-Deutsche Sammlung von Mikroorganismen und Zellkulturen GmbH, Inhoffenstr. 7 B, D-38124 Braunschweig, Germany, on 23 Apr. 2008 by Chr. Hansen A/S. This strain is well known to the person skilled in the art, and is commercially available from Chr. Hansen A/S.

### Method:

Lactococcus lactis DSM 21404 was grown by fermentation in modified Difco™ M17 Broth (Becton, Dickinson and Company, France). The modified M17 Broth contains pancreatic digest of casein 5 g/L, soy peptone 5 g/L, beef extract 5 g/L, yeast extract 2.5 g/L, ascorbic acid 0.5 g/L, magnesium sulfate 0.25 g/L, disodium-β-glycerophosphate 19 g/L and lactose 25 g/L in purified water. The inoculum for fermentation was prepared by growing the strain in a closed bottle with modified M17 Broth, under static conditions and without pH control at 30° C. The incubation period was 16 hours. Fermentation was initiated by inoculation of 1% of pre-culture to a bottle with modified M17 Broth. Fermentation was conducted under the same conditions as described for cultivation of the pre-culture. Fermentation was terminated after 16 hours. Fermentation broth was divided into aliquots for adjustment of pH of fermentation broth in the post-fermentation step. pH was adjusted with 12% ammonia water to either 5.0; or 5.5; or 6.0; or 6.5; or 7.0; or 7.5 or 8.0. Subsequently, aliquots of pH adjusted fermentation broths were cooled down to 4° C. and processed by one-step centrifugation at 4° C. in a laboratory centrifuge to produce cell concentrates. Cell concentrates were prepared by 26× concentration of the pH adjusted fermentation broth. Each cell concentrate was used for production of corresponding frozen pellets by freezing of droplets of cell concentrates in liquid nitrogen.

### Results:

Fermentation broth of L. lactis DSM 21404 was characterized by having a pH of 4.59 and OD₆₀₀ nm of 3.4. Enumeration of cells was done by flow cytometry and showed 5.04E+09 Total cells/g fermentation broth, 4.66E+09 Active cells/g fermentation broth, and the ratio of (Active cells counts/g)/(Total cell counts/g) was equal to 92.4%.

Cell counts were quantified in all frozen pellets (Table 2). Similar counts of total cells showed that the products were homogenous. Increasing flow cytometric activity of cells in frozen pellets along with increasing pH of post fermentation broths revealed that neutralization of the fermentation broth improved activity of cells upon freezing the cell concentrate in liquid nitrogen.

TABLE 2
Cell counts in frozen pellets of L. lactis DSM 21404 produced
from fermentation broths with different pH. Results are presented
as mean of quadruples with CV in parentheses.

| pH of post | | | |
|---|---|---|---|
| fermentation | Total cells/g | Active cells/g | Active cells/ |
| broth | frozen pellets | frozen pellets | Total cells (%) |
| 4.6 | 1.28E+11 (0.042) | 9.63E+10 (0.069) | 75.4 |
| 5.0 | 1.25E+11 (0.055) | 1.04E+11 (0.031) | 82.7 |
| 5.5 | 1.31E+11(0.127) | 1.04E+11 (0.078) | 79.1 |
| 6.0 | 1.29E+11 (0.029) | 1.08E+11 (0.042) | 83.6 |
| 6.5 | 1.28E+11(0.029) | 1.04E+11 (0.045) | 81.7 |
| 7.0 | 1.28E+11 (0.019) | 1.10E+11 (0.016) | 86.4 |
| 7.5 | 1.33E+11 (0.04) | 1.19E+11 (0.037) | 89.5 |
| 8.0 | 1.33E+11 (0.04) | 1.25E+11 (0.018) | 94 |

### Conclusion:

This example demonstrates that increasing the pH of bacterial cultures after termination of fermentation, before concentrating and freezing the bacterial culture, resulted in the maintenance of an increased proportion of active cells in the frozen bacterial culture.

### Example 2: Increased activity of Ligilactobacillus animalis cultures with post-fermentation pH increase

This Example involves the culture and downstream processing of a Ligilactobacillus animalis strain (formally known as Lactobacillus animalis) deposited under accession number DSM 33570 at Leibniz Institute DSMZ-German Collection of Microorganisms and Cell Cultures, Inhoffenstr. 7 B, D-38124 Braunschweig, Germany, on 8 Jul. 2020 by Chr. Hansen A/S.

### Method:

Ligilactobacillus animalis DSM 33570 was grown in a complex fermentation medium by a batch fermentation process with pH control at pH set point 5.5. Fermentation was terminated when cells reached stationary growth phase and all sugar was utilized. Fermentation broth with pH 5.5 (i.e. the bacterial culture after termination of fermentation) was divided into four aliquots and the following post-fermentation treatments were done: (1) adjustment of pH to 4.0 by addition of phosphoric acid; (2) no pH adjustment, i.e. maintaining the pH at 5.5; (3) adjustment of pH to 7.0 by addition of base, ammonium hydroxide; and (4) adjustment of pH to 8.5 by addition of base, ammonium hydroxide. Post-fermentation broths were centrifuged by one-step centrifugation in a laboratory centrifuge and cell concentrates were produced. Cell concentrates were formulated with additives containing cryo- and lyo-protective compounds as well as antioxidant. Formulated cell concentrates were processed to frozen granulates by pelletizing in liquid nitrogen. Frozen granulates were freeze-dried to freeze-dried granulates.

### Results:

Flow cytometry was used for quantification of cells and for determination of active cells in post-fermentation broth, intermediate products and final freeze-dried granulates (Table 3). Products at specific process steps were found to be similar in parameter Total cells/g product. The only exception within the group of freeze-dried granulates was the lower total cell counts in freeze-dried granulate with post-fermentation broth pH 4.0. Analysis of activities of cells across the process showed that optimum for recovery of active cells in products was obtained with post-fermentation broth with pH 7.0. The test conditions with post-fermentation pH 5.5 and post-fermentation pH 8.5 were correlated with reduced activity of cells in intermediate and final products. Adjustment of pH to 4.0 in post-fermentation broth was detrimental to the activity of cells.

TABLE 3
Flow cytometry analysis of L. animalis DSM 33570 in process
from post-fermentation broth to freeze-dried granulates

| | Post-fermentation | | | | | | Freeze-dried | |
|---|---|---|---|---|---|---|---|---|
| | broth | | Concentrate | | Frozen granulate | | granulate | |
| | | (Active | | (Active | | (Active | | (Active |
| | | cells/g)/ | | cells/g)/ | | cells/g)/ | | cells/g)/ |
| Post- | | (Total | | (Total | | (Total | | (Total |
| fermentation | Total | cells/g) | Total | cells/g) | Total | cells/g) | Total | cells/g) |
| pH | cells/g | (%) | cells/g | (%) | cells/g | (%) | cells/g | (%) |
| 4.0 | 1.94E+10 | 0 | 3.28E+11 | 1 | 1.57E+11 | n.a. | 5.96E+11 | 4 |
| 5.5 | 2.15E+10 | 88 | 3.50E+11 | 75 | 1.85E+11 | 83 | 6.48E+11 | 53 |
| 7.0 | 2.09E+10 | 95 | 3.09E+11 | 93 | 1.75E+11 | 93 | 7.43E+11 | 64 |
| 8.5 | 2.01E+10 | 91 | 3.51E+11 | 73 | 1.92E+11 | 89 | 7.46E+11 | 52 |

### Conclusion:

This example demonstrates that increasing the pH of bacterial cultures to pH 7.0 after termination of fermentation, before concentrating, formulating with protective compounds, freezing and freeze-drying the bacterial culture, resulted in the maintenance of an increased proportion of active cells at each step in the process.

### Example 3: Increased Viability and Storage Stability of Lactobacillus acidophilus Cultures with Post-Concentration pH Increase

This Example involves the culture and downstream processing of a strain of Lactobacillus acidophilus.

### Method:

A strain of Lactobacillus acidophilus was grown in a complex fermentation medium by a batch fermentation process with pH control at pH set point<5.0. Fermentation was terminated when cells reached stationary growth phase and all sugar was utilized. Fermentation broth with pH<5.0 (i.e. the bacterial culture after termination of fermentation) was centrifuged in one step in an industrial centrifuge and cell concentrate was produced. The dry matter of the cell concentrate was 18%. Cell concentrate was divided into two aliquots and the following treatments were done: (1) no pH adjustment, i.e. keeping pH<5.0; (2) adjustment of pH to 6.5 by addition of base, ammonium hydroxide. Cell concentrate with pH<5.0 contained 2E+11 Total cells/g with 85% active cells/g and cell concentrate with pH=6.5 contained 2E+11 Total cells/g with 88% Active cells/g. Both concentrates were formulated with additives containing cryo- and lyo-protective compounds as well as antioxidant. Formulated cell concentrates were processed to frozen granulates. Frozen granulates were freeze-dried to freeze-dried granulates.

### Results:

Cell counts were analysed in freeze-dried products by flow cytometry and CFU (Table 4). pH adjustment of cell concentrate to pH 6.5 showed equal flow cytometric activity of cells in freeze-dried product as in freeze-dried product made from concentrate with pH<5.0. However, the CFU analysis indicated better survival of freeze-drying and higher cell viability when freeze-dried product was made from cell concentrate with pH 6.5.

TABLE 4
Cell counts in freeze-dried products with Lactobacillus acidophilus.
Results are presented as mean of quadruples with CV in parentheses.

| | | | | (Active | |
|---|---|---|---|---|---|
| | | | | cells/g)/ | |
| | | | | (Total | |
| | | Total | Active | cells/g) | |
| Product | a_{w} | cells/g | cells/g | (%) | CFU/g |
| Freeze-dried | <0.15 | 6.96E+11 | 4.99E+11 | 72 | 2.06E+11 |
| granulate from | | (0.023) | (0.023) | | |
| concentrate with | | | | | |
| pH < 5.0 | | | | | |
| Freeze-dried | <0.15 | 6.84E+11 | 4.91E+11 | 72 | 2.46E+11 |
| granulate from | | (0.023) | (0.02) | | |
| concentrate with | | | | | |
| pH = 6.5 | | | | | |

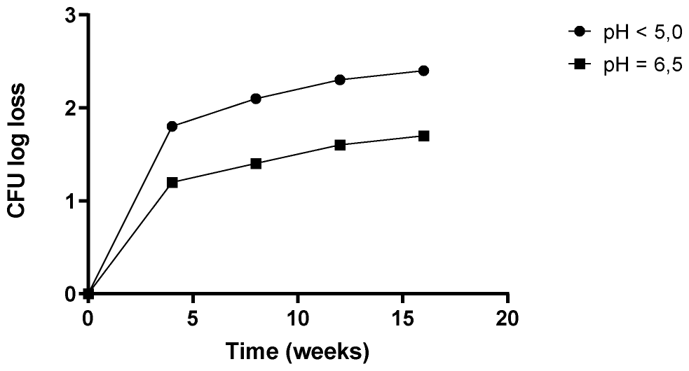

Freeze-dried granulates were sealed in aluminium pouches and tested for storage stability at 30° C. Viability of cells in freeze-dried granulates was measured by CFU analysis during 16 weeks of stability trial (FIG. 1). In addition to starting with a higher cell viability as determined by CFU (see Table 4 above), the freeze-dried product made from cell concentrate with pH 6.5 was also clearly more stable than freeze-dried product made from cell concentrate with pH<5.0 (FIG. 1).

Hence, although the proportion of active cells as measured by flow cytometry was the same for both test conditions before storage, the CFU analysis indicated better survival of freeze-drying and higher cell viability when freeze-dried product was made from cell concentrate with pH 6.5.

### Conclusion:

This example demonstrates that increasing the pH of a bacterial culture after the concentration step, before freezing and freeze-drying the bacterial culture, resulted in an increased viability count and increased storage stability of the freeze-dried product.

### Example 4: Increased Storage Stability of Bifidobacterium animalis Subsp. Lactis Cultures with Post-Fermentation pH Increase

This Example involves the culture and downstream processing of Bifidobacterium animalis subsp. lactis strain deposited under accession number DSM 33868 at Leibniz Institute DSMZ-German Collection of Microorganisms and Cell Cultures, Inhoffenstr. 7 B, D-38124 Braunschweig, Germany, on 26 May 2021 by Chr. Hansen A/S.

### Method:

Bifidobacterium animalis subsp. lactis DSM 33868 was grown in a complex fermentation medium by a batch fermentation process with pH control at pH set point 6.0±0.1. Fermentation was terminated when cells reached stationary growth phase and all sugar was utilized. Fermentation broth with pH 6.0±0.1 (i.e. the bacterial culture after termination of fermentation) was divided into five aliquots and the following post-fermentation treatments were conducted: (1) pH adjustment to pH 4.0 with phosphoric acid; (2) pH adjustment to pH 5.0 with phosphoric acid; (3) no pH adjustment, i.e. keeping pH at pH 6.0; (4) pH adjustment to pH 7.0 with base, sodium hydroxide; and (5) pH adjustment to pH 8.0 with base, sodium hydroxide. Post-fermentation broths were processed to cell concentrates, cell concentrates were formulated with additives and subsequently frozen to frozen granulates. Frozen granulates were freeze-dried to freeze-dried granulates. Freeze-dried products were transferred to aluminium bags and incubated in open aluminium bags, i.e. exposed to atmospheric air, at 30° C. and a relative humidity of 30%.

### Results:

Viability of B. animalis subsp. lactis DSM 33868 during the storage stability study was measured by CFU analysis for a period of 24 weeks (Table 5). Freeze-dried products made from acidic post-fermentation broths with pH 4.0 and pH 5.0 had poor storage stability. Among the freeze-dried products which were made from post-fermentation broths with pH 6.0-8.0, the pH 7.0 gave the most stable freeze-dried product.

TABLE 5
CFU counts in freeze-dried granulates of B. animalis subsp. lactis DSM 33868 during
stability study with exposure of product to atmospheric air, 30° C. and a_{w} = 0.3.

| Post-fermentation | CFU/g freeze-dried granulate | | | | | |
|---|---|---|---|---|---|---|
| broth pH | 0 weeks | 8 weeks | 12 weeks | 16 weeks | 20 weeks | 24 weeks |
| 4.0 | 6.08E+11 | 2.67E+08 | <1E+04 | not analysed | not analysed | not analysed |
| 5.0 | 6.78E+11 | 2.84E+10 | 2.15E+09 | <1E+04 | not analysed | not analysed |
| 6.0 | 6.23E+11 | 1.01E+10 | 2.75E+09 | 9.73E+07 | 6.10E+07 | 2.85E+07 |
| 7.0 | 5.98E+11 | 4.74E+10 | 7.51E+09 | 4.56E+08 | 4.41E+08 | 1.87E+08 |
| 8.0 | 4.28E+11 | 2.18E+10 | not available | 5.45E+07 | 2.04E+08 | 5.82E+07 |

### Conclusion:

This example demonstrates that increasing the pH of bacterial cultures after termination of fermentation, before concentrating, formulation, freezing and freeze-drying the bacterial culture, resulted in increased storage stability of the freeze-dried product.

### REFERENCES

Mogensen et al (2002) Bulletin of the International Dairy Federation, 377, 10-19.

### Deposits and Expert Solution

The applicant requests that a sample of the deposited microorganisms stated in Table 6 below may only be made available to an expert, until the date on which the patent is granted.

Table 6. Deposits made at a Depositary institution having acquired the status of international depositary authority under the Budapest Treaty on the International Recognition of the Deposit of Microorganisms for the Purposes of Patent Procedure: Leibniz Institute DSMZ-German Collection of Microorganisms and Cell Cultures (formerly known as DSMZ-Deutsche Sammlung von Mikroorganismen und Zellkulturen GmbH), Inhoffenstr. 7 B, D-38124 Braunschweig, Germany, by Chr. Hansen A/S.

| Strain | Accession No. | Deposit date |
|---|---|---|
| Lactococcus lactis | DSM 21404 | 23 Apr. 2008 |
| Ligilactobacillus animalis | DSM 33570 | 8 Jul. 2020 |
| Bifidobacterium animalis subsp. lactis | DSM 33868 | 26 May 2021 |

### Items

The present invention relates to the following items:

Item 1. A method of preparing a bacterial culture, the method comprising the steps of:
(a) culturing fermentative bacteria in a fermentation medium with a pH lower than pH 6.0 at the point of termination of fermentation and obtaining a bacterial culture,
(b) optionally concentrating the bacterial culture by a one-step concentration method to obtain a concentrated bacterial culture, and
(c) further optionally adding a protective compound to the concentrated bacterial culture of step (b);
   wherein the pH is adjusted to a pH in the range of pH 6.0 to pH 8.0 following step (a), (b) or (c).

Item 2. The method according to item 1, wherein said method comprises steps (a) and (b), or steps (a), (b) and (c), and further comprises the step of:
(d) freezing the concentrated bacterial culture to obtain a frozen bacterial culture.

Item 3. The method according to item 1 or 2, wherein said method further comprises the step of:
(e) removing water from said concentrated bacterial culture or frozen bacterial culture to obtain a dried bacterial culture.

Item 4. The method according to item 2 or 3, wherein said method further comprises the step of:
(f) packing said frozen bacterial culture obtained in step (d) or said dried bacterial culture obtained in step (e).

Item 5. The method according to any one of items 1 to 4, wherein step (b) is carried out by a technique selected from the group consisting of centrifugal separation, vacuum evaporation and filtration.

Item 6. The method according to any one of items 3 to 5, wherein step (e) is carried out by a technique selected from the group consisting of spray drying, spray freezing, vacuum drying, air drying, freeze drying, tray drying and vacuum tray drying.

Item 7. The method according to any one of the preceding items, wherein the bacterial culture comprises or consists of fermentative bacteria:
· from the phylum Firmicutes, such as:
   a lactic acid bacterium (LAB), preferably of a genus selected from the group consisting of Streptococcus (such as Streptococcus thermophilus), Lactococcus (such as Lactococcus lactis), Oenococcus (such as Oenococcus oeni), Leuconostoc (such as species Leuconostoc mesenteroides, Leuconostoc pseudomesenteroides), Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus; Eubacterium (such as Eubacterium limosum, Eubacterium aggregans, Eubacterium barkeri, Eubacterium lentum), Roseburia (Roseburia intestinalis, Roseburia hominis, Roseburia inulinivorans, Roseburia faecis and Roseburia cecicola), Faecalibacterium (such as species Faecalibacterium prausnitzii), Anaerostipes (such as Anaerostipes cacccae), Anaerobutyricum (such as species Anaerobutyricum hallii, Anaerobutyricum soehngenii)
   from the phylum Actinobacteria, such as genus Bifidobacterium (such as species Bifidobacterium animalis, Bifidobacterium longum, Bifidobacterium adolescentis, Bifidobacterium breve), genus Propionibacterium (such as species Propionibacterium freudenreichii), Cutibacterium (such as Cutibacteriun acnes)
   from the phylum Bacteroidetes, such as genera Bacteroides (such as species Bacteroides fragilis, Bacteroides xylanisolvens), Prevotella (such as species Prevotella copri) or Alistipes, and/or
   from the phylum Verrucomicrobia, such as an Akkermansia (such as species Akkermansia muciniphila).

Item 8. The method according to any one of the preceding items, wherein the fermentation medium comprises at least one carbohydrate.

Item 9. The method according to item 8, wherein the carbohydrate is one or more of: a monosaccharide such as glucose, fructose, galactose or mannose; a disaccharide such as sucrose, trehalose, maltose or lactose; a sugar alcohol such as inositol; a trisaccharide such as maltotriose or raffinose; an oligosaccharide such as a fructooligosaccharide or such as a maltodextrin with DE 3-20; a glucose syrup with DE 21-39; and a polysaccharide such as starch or inulin.

Item 10. The method according to any one of the preceding items, wherein the fermentation medium comprises at least one nitrogen source, such as peptones, yeast extract, one or more amino acids or one or more ammonia salts.

Item 11. The method according to any one of the preceding items, wherein the fermentation medium comprises one or more yield enhancing agents selected from the group consisting of a purine base, a pyrimidine base, a nucleoside, a nucleotide and derivatives thereof.

Item 12. The method according to any one of the preceding items, wherein the fermentation medium comprises one or more vitamins.

Item 12a. The method according to any one of the preceding items, wherein the fermentation medium comprises one or more fatty acids.

Item 13. The method according to any one of the preceding items, wherein the total concentration of the carbohydrate in the fermentation medium is 1-15% w/W, preferably 1-10% w/w.

Item 14. The method according to any one of the preceding items, wherein the protective compound is selected from the group consisting of cryoprotectants, lyoprotectants, antioxidants, nutrients, fillers, flavorants and mixtures thereof

Item 15. The method according to any one of the preceding items, wherein the concentration of the protective compound in the concentrated bacterial culture in step (c) is 5-90% w/w assessed as dry weights, preferably 25-75% w/w assessed as dry weights, more preferably 50-75% w/w assessed as dry weights.

Item 16. The method according to any one of the preceding items, wherein said fermentation in step (a) lasts from about 4 hours to about 7 days.

Item 17. The method according to any one of the preceding items, wherein said fermentation in step (a) is at a temperature of from about 25° C. to about 50° C.

Item 18. The method according to any one of the preceding items, wherein said termination of fermentation in step (a) is immediately followed by a holding period of from about 1 minute to about 10 hours.

Item 19. The method according to item 18, wherein during said holding period the bacterial culture is held at 4° C. to 45° C., preferably 5° C. to 37° C., 6° C. to 25° C., or more preferably 7° C. to 15° C.

Item 20. The method according to any one of the preceding items, wherein from the beginning to the termination of said fermentation in step (a), the pH decreases to no more than 6.0, such as no more than 5.5, no more than 5.0, no more than 4.5, no more than 4.0, no more than 3.5, no more than 3.0, and/or decreases by at least 0.1 pH units, preferably at least 0.2, 0.5, 1.0, 1.5, 2.0, 2.5, 3.0, 3.5 or 4.0 pH units.

Item 21. The method according to any one of the preceding items, wherein the pH adjustment following step (a), (b) or (c) is by adding a base selected from the group consisting of ammonium hydroxide, sodium hydroxide, potassium hydroxide and sodium carbonate.

Item 22. The method according to any one of items 3 to 21, wherein the dried bacterial culture has a water activity (a_{w}) in the range from 0.01-0.8, preferably in the range from 0.05-0.6, more preferably in the range from 0.05-0.4.

Item 23. The method according to any one of items 3 to 22, wherein the dried bacterial culture is in the form of a granulate and/or a powder.

Item 24. The method according to any one of items 2 to 23, wherein the frozen bacterial culture comprises in the range from 10⁴-10¹² CFU/g frozen bacterial culture.

Item 25. The method according to any one of items 3 to 24, wherein the dried bacterial culture comprises in the range from 10⁴-10¹³ CFU/g dried bacterial culture.

Item 26. The method according to any one of items 3 to 25, wherein the dried bacterial culture comprises a content of viable fermentative bacteria in the range from 10⁴-10¹³ CFU/g dried bacterial culture after storage for 16 weeks at 30° C. and a_{w}=0.3.

Item 27. The method according to any one of items 3 to 26, wherein the loss of viability of the dried bacterial culture as measured by CFU/g is less than 4 log units after storage for 16 weeks at 30° C. and a_{w}=0.3, preferably less than 3 log units, more preferably less than 2 log units, even more preferably less than 1 log units or less than 0.5 log units.

Item 28. A bacterial culture obtained by the method according to any one of items 1-27.

Item 29. A method for reducing viability loss and/or activity loss during freezing and/or drying of a bacterial culture, wherein the method comprises the steps of:
(a) culturing fermentative bacteria in a fermentation medium at a pH lower than pH 6.0 until termination of fermentation and obtaining a bacterial culture,
(b) concentrating the bacterial culture by a one-step concentration method to obtain a concentrated bacterial culture, and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

Item 30. A method for increasing storage stability of a frozen and/or dried bacterial culture, wherein the method comprises the steps of:
(a) culturing fermentative bacteria in a fermentation medium at a pH lower than pH 6.0 until termination of fermentation and obtaining a bacterial culture;
(b) concentrating the bacterial culture by a one-step concentration method to obtain a concentrated bacterial culture; and
(c) optionally adding a protective compound to the concentrated bacterial culture of step (b);
   wherein the pH is adjusted to a pH in the range of pH 6.0 to pH 8.0 following step (a), (b) or (c); and further comprising the steps of:
(d) freezing the concentrated bacterial culture to obtain a frozen bacterial culture; and/or
(e) removing water from said concentrated bacterial culture or frozen bacterial culture to obtain a dried bacterial culture.

1. A method of preparing a bacterial culture, comprising:
(a) culturing fermentative bacteria in a fermentation medium to obtain a bacterial culture, wherein the fermentation medium has a pH lower than 6.0 at termination of the fermentation, and
adjusting the pH to a pH from 6.0 to 8.0 after the fermentation.

2. The method according to claim 1, further comprising concentrating the bacterial culture by a one-step concentration method to obtain a concentrated bacterial culture, and one or both of:
freezing the concentrated bacterial culture to obtain a frozen bacterial culture; and
removing water from the concentrated bacterial culture or the frozen bacterial culture to obtain a dried bacterial culture.

3. The method according to claim 1, further comprising concentrating the bacterial culture by a one-step concentration method by a technique selected from centrifugal separation, vacuum evaporation, and filtration, to obtain a concentrated bacterial culture.

4. The method according to claim 2, wherein the method comprises the step of removing water, wherein the step of removing water is carried out by a technique selected from spray drying, spray freezing, vacuum drying, air drying, freeze drying, tray drying, and vacuum tray drying.

5. The method according to claim 1, wherein the bacterial culture comprises or consists of one or more fermentative bacteria selected from:
bacteria of a genus selected from Streptococcus, Lactococcus, Oenococcus, Leuconostoc, Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus; Eubacterium, Roseburia, Faecalibacterium, Angerostipes, and Anaerobutyricum;
bacteria of a genus selected from Bifidobacterium, Propionibacterium, and Cutibacterium;
bacteria of a genus selected from Bacteroides, Prevotella, and Alistipes, and
bacteria of genus Akkermansia.

6. The method according to claim 1, wherein the fermentation medium comprises one or more selected from:
(i) at least one carbohydrate;
(ii) at least one nitrogen source;
(iii) one or more yield enhancing agents selected from a purine base, a pyrimidine base, a nucleoside, a nucleotide, and derivatives thereof;
(iv) one or more vitamins; and
(v) one or more fatty acids.

7. The method according to claim 6, wherein the fermentation medium comprises at least one carbohydrate, wherein one or both of:
(i) the carbohydrate is one or more selected from a monosaccharide; a disaccharide; a sugar alcohol; a trisaccharide; an oligosaccharide; a glucose syrup with a dextrose equivalent value (DE) of 21-39; and a polysaccharide; and
(ii) the total concentration of the carbohydrate in the fermentation medium is 1-15% w/w.

8. The method according to claim 1, further comprising concentrating the bacterial culture by a one-step concentration method to obtain a concentrated bacterial culture, and adding a protective compound to the concentrated bacterial culture, wherein the protective compound is one or more selected from cryoprotectants, lyoprotectants, antioxidants, nutrients, fillers, flavorants and mixtures thereof, optionally wherein the concentration of the protective compound in the concentrated bacterial culture is 5-90% w/w assessed as dry weights.

9. The method according to claim 1, wherein one of more of:
(i) said fermentation lasts from about 4 hours to about 7 days;
(ii) said fermentation is at a temperature of from about 25° C. to about 50° C.;
(iii) immediately the method further comprises, following the termination of the fermentation, implementing a holding period of from about 1 minute to about 10 hours, optionally wherein during said holding period the bacterial culture is held at 4° C. to 20° C.;
and
(iv) from the beginning to the termination of the fermentation, the pH of the fermentation medium decreases to no more than 5.5.

10. The method according to any claim 1, wherein adjusting the pH comprises adding a base selected from ammonium hydroxide, sodium hydroxide, potassium hydroxide, and sodium carbonate.

11. The method according to claim 2, wherein the method comprises removing water from the concentrated bacterial culture or the frozen bacterial culture to obtain a dried bacterial culture, wherein the dried bacterial culture exhibits one or more of the following properties:
(i) has a water activity (a_{w}) in of 0.01-0.8;
(ii) is in the form of a granulate or a powder
(iii) comprises in 10⁴-10¹³ colony forming units/g (CFU/g) dried bacterial culture;
(iv) comprises a content of viable fermentative bacteria of 10⁴-10¹³ CFU/g dried bacterial culture after storage for 16 weeks at 30° C. and a_{w}=0.3; and
(v) exhibits a loss of viability of less than 4 log units as measured by CFU/g after storage for 16 weeks at 30° C. and a_{w}=0.3.

12. The method according to claim 2, wherein the method comprises freezing the concentrated bacterial culture to obtain a frozen bacterial culture, wherein the frozen bacterial culture comprises in the range from 10⁴-10¹² CFU/g frozen bacterial culture.

13. A bacterial culture obtained by the method according to claim 1.

14. A method for reducing viability loss and/or activity loss during freezing and/or drying of a bacterial culture, comprising:
(a) culturing fermentative bacteria in a fermentation medium to obtain a bacterial culture, wherein the fermentation medium has a pH lower than 6.0 at termination of the fermentation,
(b) concentrating the bacterial culture by a one-step concentration method to obtain a concentrated bacterial culture,
(c) optionally, adding a protective compound to the concentrated bacterial culture of step (b);
(d) following step (a), (b), or (c) (if conducted), adjusting the pH to a range of pH 6.0 to pH 8.0;
and one or both of:
(e) freezing the concentrated bacterial culture to obtain a frozen bacterial culture; and
(f) removing water from the concentrated bacterial culture or the frozen bacterial culture to obtain a dried bacterial culture.

15. A method for increasing storage stability of a frozen and/or dried bacterial culture, comprising:
(a) culturing fermentative bacteria in a fermentation medium to obtain a bacterial culture, wherein the fermentation medium has a pH lower than 6.0 at termination of the fermentation;
(b) concentrating the bacterial culture by a one-step concentration method to obtain a concentrated bacterial culture;
(c) optionally, adding a protective compound to the concentrated bacterial culture of step (b);
(d) following step (a), (b), or (c) (if conducted), adjusting the pH to a range of pH 6.0 to pH 8.0;
and one or both of:
(e) freezing the concentrated bacterial culture to obtain a frozen bacterial culture; and
(f) removing water from the concentrated bacterial culture or the frozen bacterial culture to obtain a dried bacterial culture.

16. The method according to claim 1, wherein the bacterial culture comprises or consists of one or more fermentative bacteria selected from:
Streptococcus thermophilus, Lactococcus lactis, Oenococcus oeni, Leuconostoc mesenteroides, Leuconostoc pseudomesenteroides, Eubacterium limosum, Eubacterium aggregans, Eubacterium barkeri, Eubacterium lentum, Roseburia intestinalis, Roseburia hominis, Roseburia imulinivorans, Roseburia faecis, Roseburia cecicola, Faecalibacterium prausnitzii, Anaerostipes cacccae, Anaerobutyricum hallii, and Anaerobutyricum soehngenii;
Bifidobacterium animalis, Bifidobacterium longum, Bifidobacterium adolescentis, Bifidobacterium breve, Propionibacterium freudenreichii, and Cutibacteriun acnes;
Bacteroides fragilis, Bacteroides xylanisolvens, and Prevotella copri, and
Akkermansia muciniphila.

17. The method according to claim 1, further comprising adding a protective compound to the concentrated bacterial culture.

18. The method according to claim 17, wherein the protective compound is one or more selected from cryoprotectants, lyoprotectants, antioxidants, nutrients, fillers, flavorants and mixtures thereof.
